(19) United States
(12) Patent Application Publication
(10) Pub. No.: US 2023/0414910 A1
(43) Pub. Date: Dec. 28, 2023

(54) HOUSING FOR BIOLOGIC MATERIALS AND OTHER SURGICAL DEVICES, AND METHODS OF USE THEREFOR

(71) Applicant: Origami Surgical, Inc., Madison, NJ (US)

(72) Inventors: Patrick John Culligan, Madison, NJ (US); John Gillespie, JR., Dover, MA (US); Sean McNerney, Naples, FL (US); Marc Beer, Boston, MA (US)

(21) Appl. No.: 18/340,478

(22) Filed: Jun. 23, 2023

Related U.S. Application Data
(63) Continuation of application No. PCT/US2021/064897, filed on Dec. 22, 2021.
(60) Provisional application No. 63/129,925, filed on Dec. 23, 2020.

(51) Int. Cl. A61M 31/00; A61L 31/04; A61L 31/14; A61B 10/04; A61B 10/02

(52) U.S. Cl. CPC A61M 31/002; A61L 31/044; A61L 31/148; A61B 10/04; A61B 10/0266; A61M 2205/32; A61M 2210/1017

(57) ABSTRACT

Various embodiments for an apparatus for delivering a biologic into a human or animal subject are disclosed. In one embodiment, an apparatus includes a housing comprising an interior chamber, the housing having a shape and size for insertion into a human or animal subject. A biologic is positioned within the interior chamber that is removable from the housing while the housing is inserted into the human or animal subject.

### CROSS-REFERENCE TO RELATED APPLICATION

This application is a continuation of and claims priority to International Application No. PCT/US2021/064897, filed Dec. 22, 2021, which claims the benefit of and priority to U.S. Provisional Application No. 63/129,925 entitled “HOUSING FOR BIOLOGIC MATERIALS AND OTHER SURGICAL DEVICES, AND METHODS OF USE THEREFOR,” filed Dec. 23, 2020, all of the contents of which being incorporated by reference in their entirety herein.

### BACKGROUND

Many laparoscopic surgeries are performed using robotic technology, such as the DA VINCI® surgical system. One such procedure that can be performed with the use of the DA VINCI® surgical system is a sacrocolpopexy, which is a surgical procedure to correct vaginal vault prolapse. During this procedure, the surgeon positions mesh to support the vagina. In so doing, a surgeon does a lot of suturing. It is not uncommon for a surgeon to use 16-24 separate sutures in a single procedure. The process of suturing is quite time-consuming and requires a very skilled assistant to pass the needles, each carrying a suture, to the surgeon controlling the robotic arm. Also typically, such needles are passed to the surgeon within the patient's abdomen. Thus, the assistant must be skilled and highly trained so as to avoid nicking an artery or other tissue while passing the needles.

U.S. Pat. No. 6,986,780 B2 discloses a surgical element carrier and method for use. The surgical element carrier includes a housing dimensioned to contain therein surgical elements, and dimensioned for insertion through a surgical port used in minimally invasive surgeries. The housing further includes a first housing portion and a second housing portion movable relative to the first housing portion between a closed position wherein the surgical elements are substantially surrounded by the housing, and an open position where the surgical elements are at least partially exposed and removable from the housing.

### TECHNICAL FIELD

The present disclosure relates generally to the field of medical devices, and in particular, to an apparatus for housing biologics (e.g., in a liquid, solid, or powdered form), sponges, and other surgical devices (e.g., needles), and methods of use therefor.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIGS. 1-11 are various view of an apparatus for housing biologics and/or surgical instruments according to various embodiments of the present disclosure.

### DETAILED DESCRIPTION

The present disclosure may be understood more readily by reference to the following detailed description of the disclosure taken in connection with the accompanying drawing figures, which form part of this disclosure. It is also understood that this disclosure is not limited to the specific devices, methods and conditions or parameters described and/or shown herein, and that the terminology used herein is for the purpose of describing particular embodiments by way of example only and is not intended to be limiting of the claimed disclosure. Also, as used in the specification including the appended claims, the singular forms “a,” “an,” and “the” include the plural, and reference to a particular numerical value indicates at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

The present disclosure provides canisters or apparatuses for housing a plurality of biologics (e.g., liquid, powder, or solid biologics), surgical elements (e.g., needles with associated/threaded sutures), and other components which may be useful in surgical and/or robot-assisted surgical procedures. Such canisters can be used with robotic surgical technology, such as with the DA VINCI® surgical system, and can be placed and manipulated inside the body of a human or animal subject.

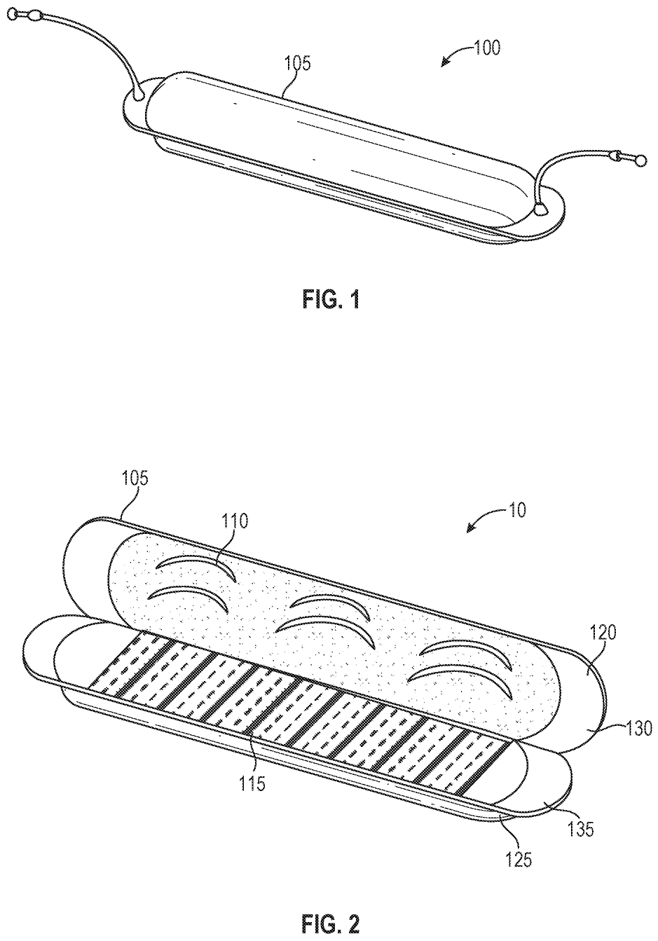

Referring to FIGS. 1-10, various embodiments for an apparatus 100 are shown. The apparatus 100 may include a generally cylindrical, substantially-rigid housing 105 that is approximately 5 cm long and 11 mm in diameter. The housing may have a diameter or largest cross-sectional dimension that is less than about 12 mm, which allows the housing to be inserted into a typical surgical incision or portion for laparoscopic surgery or other suitable procedure. However, it is understood that the disclosure is not limited to these dimensions, and other suitable dimensions may be employed.

The housing 105 may be sized and dimensioned to contain therein a plurality of surgical elements 110 and/or a biologic 115. The housing 105 may be further sized and dimensioned for insertion through a surgical port, such as a surgical port used in minimally invasive surgeries. In various embodiments, the housing 105 may include a clamshell housing or, in other words, a first housing portion 120 and a second housing portion 125 hingedly connected to and pivotable or otherwise movable relative to the first housing portion between a closed position, where the surgical elements 110 are substantially surrounded by the housing 105, and an open position where the surgical elements 110 are at least partially exposed and removable from the housing 105. The first housing portion 120 and/or the second housing portion 125 may include tabs 130, 135 that are located at distal ends of the housing 105 that project outwardly. The tabs 130, 135 may be positioned on one or both ends of the housing 105.

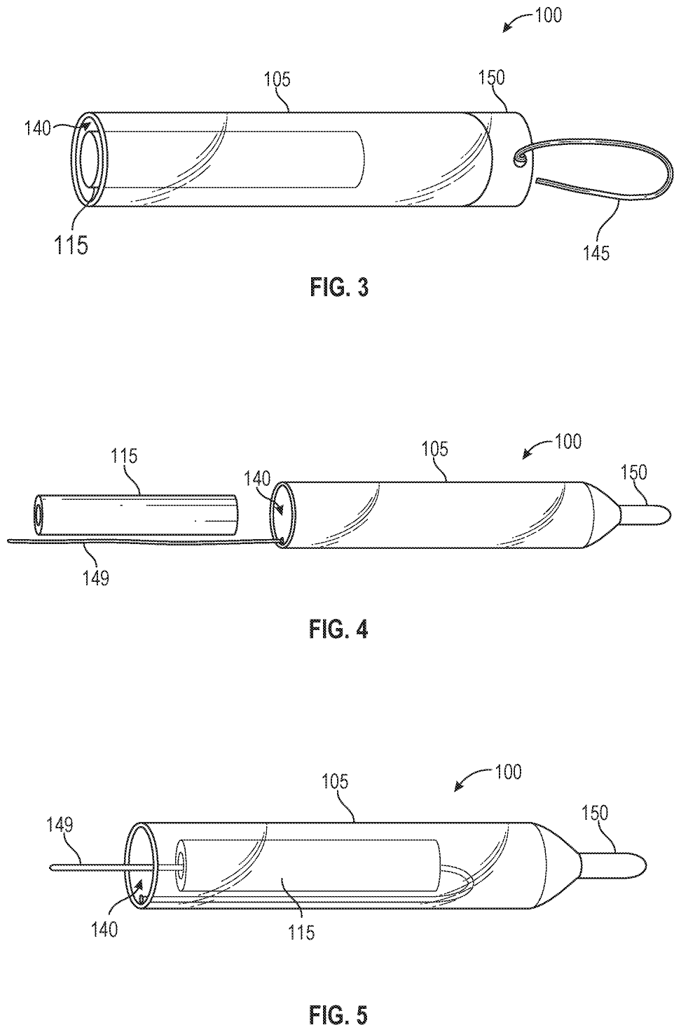

Referring now to FIGS. 3-5, an embodiment of the apparatus 100 is shown in which the housing 105 includes an interior chamber 140. Like the embodiment of FIGS. 1 and 2, the housing 105 may have a size and shape suitable for insertion into a human or animal subject. A biologic 115 may be positioned within the interior chamber 140 that is removable from the housing 105, for instance, while the housing 105 is inserted into the human or animal subject. The biologic 115, while referred to herein in the singular, may include one or more biologics 115 (or, in other words, at least one biologic). A draw string 145 may be attached to the housing 105. In some embodiments, the draw string 145 may be formed of stainless steel wire or other metallic and biocompatible material. One end of the housing 105 is open, or comprises an aperture, such that the biologic 115 may be inserted into and removed from the housing 105. In some embodiments, the housing 105 includes a removable cap (not shown) configured to form an interference, snap, friction, magnetic, threaded, or other suitable connection to the housing 105 to fully encapsulate the biologic 115.

According to various embodiments, the biologic 115 may include one of a solid compound; a liquid compound; and a gaseous compound. In some embodiments, the biologic 115 comprises a material pre-treated with and containing at least one of: bupivacaine and bupivacaine hydrochloride. For example, in some embodiments, the biologic 115 comprises a biocompatible cloth or biocompatible sponge material pre-treated with and containing at least one of: bupivacaine and bupivacaine hydrochloride. The biologic 115 may be stored in the apparatus 100 in a rolled arrangement, a flat arrangement, or other suitable arrangement. The material of the biologic 115 may further include a collagen-based implant material that is fully or partially bioresorbable. The biologic 115 may be removed from the interior chamber using a draw string 149 (FIG. 4), a finger, a robotic manipulator, or other suitable mechanism. To this end, the biologic 115 may be coupled to the draw string 149, or the body of the biologic 115 may include a tab or other suitable gripping portion.

The housing 105 of the apparatus 100 may include a first distal end, where the first distal end comprises an opening to the interior chamber 140 through which the biologic 115 is retrievable, and a second distal end. The second distal end may not include any opening (e.g., a closed distal end) and may include a tab portion 150, such as a flattened tab portion 150. As noted above, in some embodiments, the first distal end may include a cap (not shown) detachably attachable to the first distal end. The cap may be removable as a slide off cap or a twisting cap that utilizes a threaded connection in some embodiments.

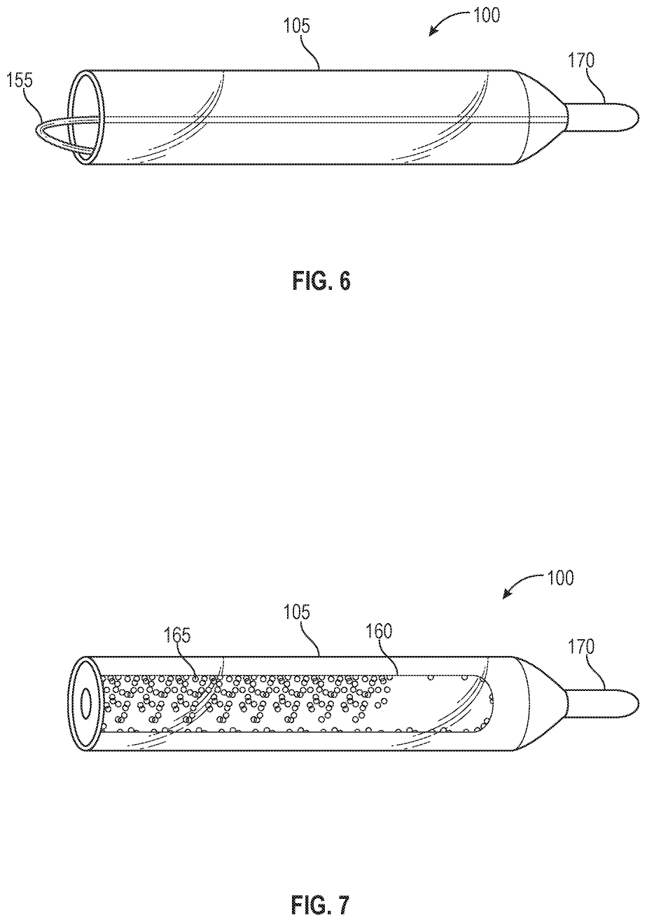

Referring now to FIGS. 6 and 7, another embodiment of the apparatus 100 is shown. The housing 105 includes a uniform portion and an exposed portion. A first tab 155 is positioned on a first end of the apparatus 100 coupled to the housing 105. A storage device 160 is positioned within the housing 105. The storage device 160 may, like the housing 105, include a generally elongated and/or cylindrical body having a cross-section size less than that of the housing 105. In some embodiments, the storage device 160 is configured to rotate relative to the housing 105. The storage device 160 may include an interior chamber having a biologic 115 stored therein, for instance, in a powered state, liquid state, or other suitable state. The storage device 160 may further include one or more apertures 165.

A second tab 170 may be positioned on a second end of the apparatus 100 and/or housing 105 that is engaged with or coupled to the storage device 160. A rotation of the first tab 155 relative to the second tab 170 may cause the storage device 160 contained in the housing 105 to rotate relative to the housing 105, such that the biologic 115 is dispensable when the at least one aperture 165 of the storage device 45 is aligned with an exposed portion of the housing 105 (e.g., a window notched in a body of the housing 105). For instance, when the biologic 115 is in the powered form or liquid form, an operator of the apparatus 100 can deliver the biologic through the at least one aperture 165 of the storage device 160 and the exposed portion of the housing 105 upon an agitation of the apparatus 100 (e.g., shaking).

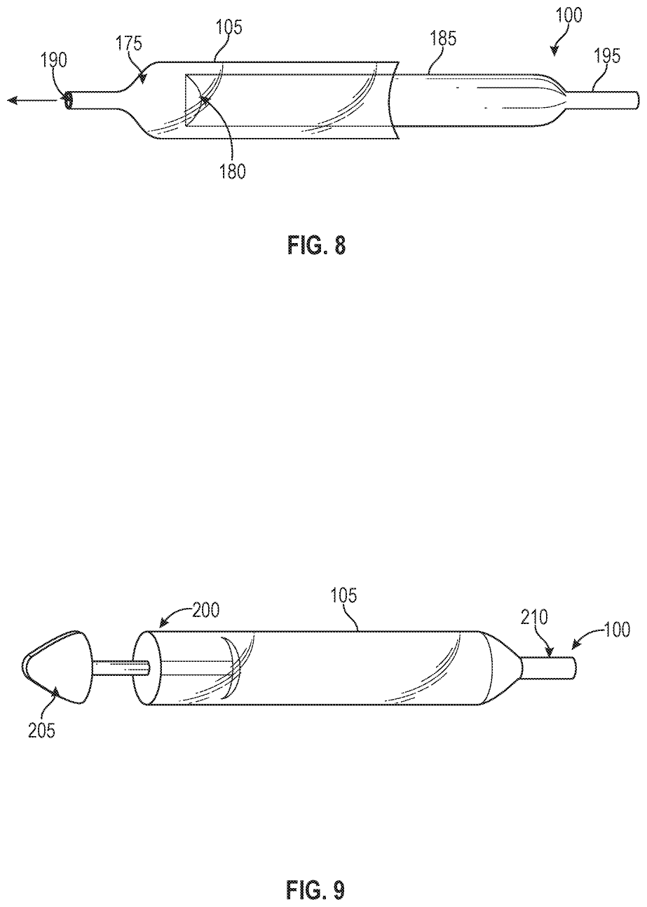

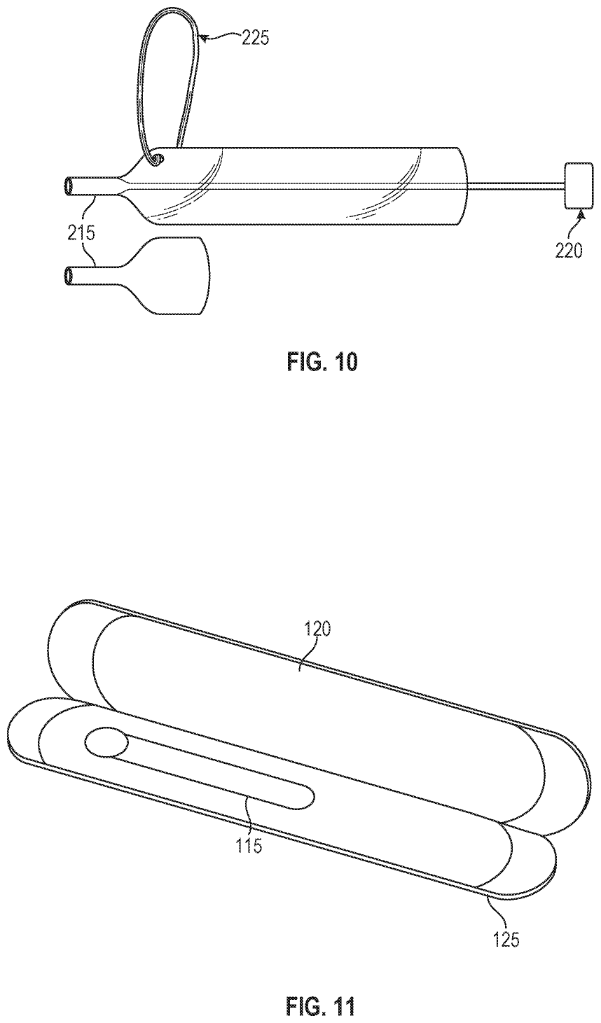

Turning next to FIGS. 8 and 9, another embodiment for the apparatus 100 is shown for delivering a biologic 115 into a human or animal subject is shown, comprising a housing 105. The housing 105 acts a syringe by having an interior chamber 175 comprising a biologic 115 positioned or stored therein, a stopper 180, and a plunger 185. A first distal end of the housing 105 includes a dispensing orifice 190 through which the biologic 115 (e.g., in liquid or semi-liquid form) is dispensed based on an actioning of the plunger 185 relative to the stopper 180. The housing 105 further include a second distal end comprising a flat tab portion 195.

Referring specifically to FIG. 9, the embodiment shown in FIG. 9 includes the apparatus 100 having a syringe 200 with a no-thumb flange 205. The no-thumb flange 205 may include a flat area a robotic manipulator may grab. In some embodiments, the no-thumb flange 205 includes a semi-circular body, but may include a square or rectangular-shaped body in some examples. The apparatus 100 further includes a dispensing tube 210, which may be sized and positioned such that a long robot can capture.

Moving along to FIG. 10, another embodiment of the apparatus 100 is shown according to various embodiments. The apparatus 100 includes a dispensing tip 215 comprising an aperture through which a liquid or semi-liquid compound (e.g., the biologic 115) may be forced. The apparatus 100 may be configured to connect to an external syringe via a luer lock 220 or other suitable connection mechanism. The apparatus 100 may include an elongated tube fluidly coupled to the dispensing tip 215 and the luer lock 220. In some embodiments, the apparatus 100 includes a retrieval band 225 that assists robotic manipulators or other gripping of the apparatus 100. The retrieval band 225 may be positioned through an aperture 230 in the housing 105 of the apparatus 100.

Referring now to FIG. 11, in some embodiments, the housing 105 is a clamshell housing having a first section hingedly connected to a second section, like the embodiment shown in FIGS. 1 and 2. In other words, the apparatus 100 includes a first housing portion 120 and a second housing portion 125 pivotable or otherwise movable relative to the first housing portion between a closed position, where a biopsy punch 235 is substantially surrounded by the housing 105, and an open position where the biopsy punch 235 is at least partially exposed and removable from the housing 105. The biopsy punch 235 may be coupled to the second housing portion 125, for example, whereas the first housing portion 120 may include a container or hollow interior for biopsy tissue storage. The hollow interior may be accessible via a hinged door, sliding door, or other suitable mechanism, or may be otherwise open relative to the biopsy punch 235. A closing mechanism, such as a latch or hinge, may be provided for securing the housing 105 in a closed position.

Referring collectively to the figures, in some embodiments, the housing 105 may include a radiopaque element. The radiopaque element may include a radiopaque element comprising at least one of radiopaque ink, a metal wire (e.g., a stainless steel wire); and metal tape. The radiopaque element may have a Hounsfield value (HU) property of at least 1000 HU or at least 2000 H U. The biologic may further include at least one of a mesh, an anti-adhesion material, a hemostatic material, and an analgesic sponge.

According to various embodiments, a method for delivering a biologic into a human or animal subject is described, comprising: providing a housing comprising an interior chamber; positioning a biologic within the interior chamber that is removable from the housing; performing a surgical procedure in which the housing is positioned at a surgical site within a human or animal subject; and treating the surgical site within the human or animal subject using the biologic by removing the biologic from the interior chamber of the housing. The surgical procedure may include a laparoscopy. The surgical site may include a peritoneal cavity of the human or animal subject. A method may include providing one or more of the apparatuses described herein.

Although the housings 105 of many of the example embodiments are shown as generally cylindrical, any suitably shaped housing 105 can be employed and be within the scope of the present disclosure, including polygonal-shaped, box-shaped, and generally semi-cylindrically shaped. Additionally, the dimensions of the housing 105 can vary as desired (and hence the apparatus 100 can be larger or smaller) and still be within the scope of the present disclosure. Moreover, any biocompatible material can be used to construct the housing 105, including hard plastics and metals, such as stainless steel. Alternatively, a flexible or substantially non-rigid material can be used for the housing 105.

The features, structures, or characteristics described above may be combined in one or more embodiments in any suitable manner, and the features discussed in the various embodiments are interchangeable, if possible. In the following description, numerous specific details are provided in order to fully understand the embodiments of the present disclosure. However, a person skilled in the art will appreciate that the technical solution of the present disclosure may be practiced without one or more of the specific details, or other methods, components, materials, and the like may be employed. In other instances, well-known structures, materials, or operations are not shown or described in detail to avoid obscuring aspects of the present disclosure.

Although the relative terms such as “on,” “below,” “upper,” and “lower” are used in the specification to describe the relative relationship of one component to another component, these terms are used in this specification for convenience only, for example, as a direction in an example shown in the drawings. It should be understood that if the device is turned upside down, the “upper” component described above will become a “lower” component. When a structure is “on” another structure, it is possible that the structure is integrally formed on another structure, or that the structure is “directly” disposed on another structure, or that the structure is “indirectly” disposed on the other structure through other structures.

In this specification, the terms such as “a,” “an,” “the,” and “said” are used to indicate the presence of one or more elements and components. The terms “comprise,” “include,” “have,” “contain,” and their variants are used to be open ended, and are meant to include additional elements, components, etc., in addition to the listed elements, components, etc. unless otherwise specified in the appended claims. The terms “first,” “second,” etc. are used only as labels, rather than a limitation for a number of the objects.

The above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Clause 1. An apparatus for delivering a biologic into a human or animal subject, comprising: a housing comprising an interior chamber, the housing having a shape and size for insertion into a human or animal subject; and a biologic positioned within the interior chamber that is removable from the housing while the housing is inserted into the human or animal subject, wherein the biologic is one of a solid compound; a liquid compound; and a gaseous compound.

Clause 2. The apparatus of clause 1, wherein the biologic is a solid compound, and comprises a material pre-treated with and containing at least one of bupivacaine and bupivacaine hydrochloride.

Clause 3. The apparatus of any of clauses 1-2, wherein the material comprises a collagen-based implant material that is fully or partially bioresorbable.

Clause 4. The apparatus of any of clauses 1-3, wherein: the housing is elongated tubular body having a circular cross-section; the housing is a clamshell housing having a first housing portion hingedly connected to a second housing portion; and the housing comprises a closing mechanism for securing the housing in a closed position.

Clause 5. The apparatus of any of clauses 1-4, wherein the first housing section is configured to store a biopsy punch device and the second housing section is configured to store biopsy tissue.

Clause 6. The apparatus of any of clauses 1-5, wherein the housing comprises a radiopaque element, wherein the radiopaque element comprises at least one of: radiopaque ink, a metal wire; and metal tape.

Clause 7. The apparatus of any of clauses 1-6, wherein the radiopaque element comprises a Hounsfield value (HU) property of at least 1000 HU.

Clause 8. The apparatus of any of clauses 1-7, wherein the radiopaque element comprises a Hounsfield value (HU) property of at least 2000 HU.

Clause 9. The apparatus of any of clauses 1-8, wherein the housing comprises: a first distal end, the first distal end comprises an opening to the interior chamber through which he biologic is retrievable; and a second distal end, the second distal end not comprising any opening and comprises a flattened tab portion.

Clause 10. The apparatus of any of clauses 1-9, wherein the first distal end comprises a cap detachably attached to the first distal end, wherein the cap is removable as a slide off cap or a twisting cap that utilizes one of an interference, a friction, a threaded, and a magnetic connection.

Clause 11. The apparatus of any of clauses 1-10, wherein the biologic comprises at least one of: a mesh, an anti-adhesion material, a hemostatic material, and an analgesic sponge.

Clause 12. An apparatus for delivering a biologic into a human or animal subject, comprising a housing, wherein the housing comprises: an interior chamber comprising a biologic positioned thereon; a stopper; and a plunger; wherein a first distal end of the housing comprises a dispensing orifice through which the biologic is dispensed based on an actioning of the plunger relative to the stopper.

Clause 13. The apparatus of clause 12, wherein the housing comprises a second distal end comprising a flat tab portion.

Clause 14. An apparatus for delivering a biologic into a human or animal subject, comprising: a housing comprising a uniform portion and a window; a first tab on a first end of the apparatus coupled to the housing; a storage device positioned within the housing, the storage device comprising at least one aperture and being configured to rotate relative to the housing, the storage device comprising an interior chamber having a biologic in a state stored therein such that the biologic can navigate through the at least one aperture; and a second tab on a second end of the apparatus coupled to the storage device coupled to the storage device, wherein a rotation of the first tab relative to the second tab causes the storage device to rotate relative to the housing such that the at least one aperture aligns with a window and the biologic is dispensable through the window.

Clause 15. The apparatus of clause 14, wherein the biologic is in a powered or liquid form configured to be delivered through the at least one aperture of the storage device and the window of the housing upon an agitation of the apparatus.

Clause 16. A method for delivering a biologic into a human or animal subject, comprising: providing a housing comprising an interior chamber; positioning a biologic within the interior chamber that is removable from the housing; performing a surgical procedure in which the housing is positioned at a surgical site within a human or animal subject; and treating the surgical site within the human or animal subject using the biologic by removing the biologic from the interior chamber of the housing.

Clause 17. The method of clause 16, wherein the surgical procedure comprises a laparoscopy.

Clause 18. The method of clause 16, wherein the surgical site comprises a peritoneal cavity of the human or animal subject.

Clause 19. A method comprising providing the apparatus(es) of clauses 1-18.

Therefore, the following is claimed:

1. An apparatus for delivering a biologic into a human or animal subject, comprising:
a housing comprising an interior chamber, the housing having a shape and size for insertion into a human or animal subject; and
a biologic positioned within the interior chamber that is removable from the housing while the housing is inserted into the human or animal subject, wherein the biologic is one of a solid compound; a liquid compound; and a gaseous compound.

2. The apparatus of claim 1, wherein the biologic is a solid compound, and comprises a material pre-treated with and containing at least one of bupivacaine and bupivacaine hydrochloride.

3. The apparatus of claim 2, wherein the material comprises a collagen-based implant material that is fully or partially bioresorbable.

4. The apparatus of claim 1, wherein:
the housing is elongated tubular body having a circular cross-section;
the housing is a clamshell housing having a first housing portion hingedly connected to a second housing portion; and
the housing comprises a closing mechanism for securing the housing in a closed position.

5. The apparatus of claim 4, wherein the first housing section is configured to store a biopsy punch device and the second housing section is configured to store biopsy tissue.

6. The apparatus of claim 1, wherein the housing comprises a radiopaque element, wherein the radiopaque element comprises at least one of: radiopaque ink, a metal wire; and metal tape.

7. The apparatus of claim 6, wherein the radiopaque element comprises a Hounsfield value (HU) property of at least 1000 HU.

8. The apparatus of claim 6, wherein the radiopaque element comprises a Hounsfield value (HU) property of at least 2000 HU.

9. The apparatus of claim 1, wherein the housing comprises:
a first distal end, the first distal end comprises an opening to the interior chamber through which the biologic is retrievable; and
a second distal end, the second distal end not comprising any opening and comprises a flattened tab portion.

10. The apparatus of claim 1, wherein the first distal end comprises a cap detachably attached to the first distal end, wherein the cap is removable as a slide off cap or a twisting cap that utilizes one of an interference, a friction, a threaded, and a magnetic connection.

11. The apparatus of claim 1, wherein the biologic comprises at least one of: a mesh, an anti-adhesion material, a hemostatic material, and an analgesic sponge.

12. An apparatus for delivering a biologic into a human or animal subject, comprising a housing, wherein the housing comprises:
an interior chamber comprising a biologic positioned thereon;
a stopper; and
a plunger;
wherein a first distal end of the housing comprises a dispensing orifice through which the biologic is dispensed based on an actioning of the plunger relative to the stopper.

13. The apparatus of claim 12, wherein the housing comprises a second distal end comprising a flat tab portion.

14. An apparatus for delivering a biologic into a human or animal subject, comprising:
a housing comprising a uniform portion and a window;
a first tab on a first end of the apparatus coupled to the housing;
a storage device positioned within the housing, the storage device comprising at least one aperture and being configured to rotate relative to the housing, the storage device comprising an interior chamber having a biologic in a state stored therein such that the biologic can navigate through the at least one aperture; and
a second tab on a second end of the apparatus coupled to the storage device coupled to the storage device, wherein a rotation of the first tab relative to the second tab causes the storage device to rotate relative to the housing such that the at least one aperture aligns with a window and the biologic is dispensable through the window.

15. The apparatus of claim 14, wherein the biologic is in a powered or liquid form configured to be delivered through the at least one aperture of the storage device and the window of the housing upon an agitation of the apparatus.

16. A method for delivering a biologic into a human or animal subject, comprising:
providing a housing comprising an interior chamber;
positioning a biologic within the interior chamber that is removable from the housing;
performing a surgical procedure in which the housing is positioned at a surgical site within a human or animal subject; and
treating the surgical site within the human or animal subject using the biologic by removing the biologic from the interior chamber of the housing.

17. The method of claim 16, wherein the surgical procedure comprises a laparoscopy.

18. The method of claim 16, wherein the surgical site comprises a peritoneal cavity of the human or animal subject.
